(19) United States
(12) Patent Application Publication
(10) Pub. No.: US 2023/0389864 A1
(43) Pub. Date: Dec. 7, 2023

(54) IDENTIFICATION OF A NOCICEPTION PARAMETER

(71) Applicant: Covidien LP, Mansfield, MA (US)

(72) Inventors: Paul S. Addison, Edinburgh (GB); Dean Montgomery, Edinburgh (GB)

(21) Appl. No.: 18/249,945

(22) PCT Filed: Nov. 18, 2021
(86) PCT No.: PCT/US2021/059909

§ 371 Date: Apr. 20, 2023

Related U.S. Application Data
(60) Provisional application No. 63/116,633, filed on Nov. 20, 2020.

(51) Int. Cl. A61B 5/00; G16H 50/30

(52) U.S. Cl. CPC A61B 5/4821; A61B 5/7282; A61B 2505/05; G16H 50/30; A61B 5/4839

(57) ABSTRACT

In some examples, a patient monitoring system includes processing circuitry configured to detect an occurrence of a nociception event of a patient during a medical procedure. The processing circuitry may, for example, monitor a nociception parameter of the patient during the medical procedure, determine a characteristic nociception parameter at a point in time based at least in part on values of the nociception parameter over a period of time, and determine, based at least in part on comparing the characteristic nociception parameter at the point in time with a nociception threshold, a nociception event has occurred at the point in time. In some examples, the processing circuitry is configured to provide an indication to adjust an amount of analgesic administered to the patient based on the determination that the nociception event has occurred at the point in time.

This application claims priority from U.S. Provisional Patent Application No. 63/116,633, filed on Nov. 20, 2020, the entire content of which is incorporated herein by reference.

### TECHNICAL FIELD

This disclosure relates to patient monitoring.

### BACKGROUND

Nociception is a response of a sensory nervous system of a subject to certain stimuli, such as chemical, mechanical, or thermal stimuli, that causes the stimulation of sensory nerve cells called nociceptors.

### SUMMARY

The present disclosure describes example devices, systems, and techniques for monitoring the nociception parameters of a patient undergoing a medical procedure based on one or more changes in the nociception parameter over time. A clinician may use a nociception monitoring system to monitor the nociception parameters of the patient during a medical procedure to help determine an amount of analgesic to administer to the patient during the medical procedure (e.g., a quantity and/or a time at which to deliver the analgesic).

In accordance with aspects of the present disclosure, instead of determining whether a patient is experiencing a severe nociceptive stimulus based solely on whether the nociception parameter of the patient has increased to be greater than or equal to a nociception threshold, a nociception monitoring system determines a characteristic nociception parameter based at least in part on values of the nociception parameter over a period of time. For example, the system can be configured to determine a characteristic nociception parameter as an average of the values of the nociception parameter over the period of time or as a weighted average of the values of the nociception parameter over the period of time. The clinician may therefore compare the characteristic nociception parameter to the nociception threshold to determine whether the patient is experiencing a severe nociceptive stimulus.

In one aspect, a method includes monitoring, by processing circuitry, a nociception parameter of a patient during a medical procedure; determining, by the processing circuitry, a characteristic nociception parameter at a point in time based at least in part on values of the nociception parameter over a period of time; determining, by the processing circuitry and based at least in part on comparing the characteristic nociception parameter at the point in time with a nociception threshold, a nociception event has occurred at the point in time; and providing, by the processing circuitry, an indication to adjust an amount of analgesic administered to the patient based on the determination that the nociception event has occurred at the point in time.

In another aspect, a system includes: memory configured to store a nociception threshold; and processing circuitry configured to: monitor a nociception parameter of a patient during a medical procedure; determine a characteristic nociception parameter at a point in time based at least in part on values of the nociception parameter over a period of time; determine, based at least in part on comparing the characteristic nociception parameter at the point in time with the nociception threshold, a nociception event has occurred at the point in time; and provide an indication to adjust an amount of analgesic administered to the patient based on the determination that the nociception event has occurred at the point in time, wherein the determination is made based on a comparison between the characteristic nociception parameter at the point in time with a nociception threshold.

In another aspect, a non-transitory computer readable storage medium comprises instructions that, when executed, cause processing circuitry to: monitor a nociception parameter of a patient during a medical procedure; determine a characteristic nociception parameter at a point in time based at least in part on values of the nociception parameter over a period of time; determine, based at least in part on comparing the characteristic nociception parameter at the point in time with a nociception threshold, a nociception event has occurred at the point in time; and provide an indication to adjust an amount of analgesic administered to the patient based on the determination that the nociception event has occurred at the point in time, wherein the determination is made based on a comparison between the characteristic nociception parameter at the point in time with a nociception threshold.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a block diagram illustrating an example environment in which a patient monitoring system monitors one or more nociception parameters of a patient undergoing a medical procedure.

FIGS. 2A-2D illustrate example techniques for determining a characteristic nociception parameter, in accordance with aspects of this disclosure.

FIG. 3 is a block diagram illustrating the patient monitoring system of FIG. 1.

FIG. 4 is a flow diagram illustrating an example method of determining whether to increase the amount of analgesic administered to patient undergoing a medical procedure.

### DETAILED DESCRIPTION

Aspects of the present disclosure describe devices, systems, and techniques for monitoring a nociception parameter of a patient undergoing a medical procedure, such as surgery, to help determine an amount of analgesic to administer to the patient during the medical procedure (e.g., a bolus of analgesic or other quantity, and/or a time at which to deliver the analgesic). In some examples, a patient monitoring system, also referred to herein as a nociception monitor or a nociception monitoring system, may provide a continuous measure of a nociception parameter for a patient undergoing a medical procedure in order to track the nociception response of the patient. The nociception parameter can be based on one or more sensed physiological signals, such as an electrocardiogram (ECG), a photoplethysmogram (PPG), electroencephalogram (EEG), skin conductance, body temperature, and the like or combinations thereof, and may typically be displayed over time.

A clinician may monitor the nociception parameter of a patient to determine the amount of analgesic to administer to the patient during the medical procedure. As the patient undergoes the medical procedure, the clinician may administer analgesic to the patient to reduce stress experienced by the patient during the medical procedure. While this stress is generally referred to herein as “surgical stress,” the stress may be the result of one or more events occurring during any medical procedure and is not limited to surgery-induced stress responses of a patient. The stress can be, for example, an activation of a patient's sympathetic nervous system, an endocrine response, and/or immunological or hematological change in the patient. Example nociception parameters include nociception level index (NOL), analgesia nociception index (ANI), surgical pleth index (SPI), composite variability index (CVI), and the like.

A clinician may use a nociception monitoring system to monitor the nociception parameter of the patient, which may correspond to the amount of surgical stress experienced by the patient, during the medical procedure, and the clinician may determine whether to adjust the amount of analgesic to administer to the patient based on the nociception parameter of the patient. In some examples, the clinician may monitor the nociception parameter of the patient to determine whether the nociception parameter of the patient increases above a nociception threshold, which may indicate a severe nociceptive stimulus experienced by the patient. The clinician may, in response to the nociception parameter of the patient increasing above the nociception threshold, adjust (e.g., increase) the amount of analgesic to dampen the nociception stimulus experienced by the patient.

Noise in the nociception parameters may occasionally cause false positive indications of a severe nociceptive stimulus. Such noise may be caused by patient motion, electrocautery, administration of drugs to the patient, and the like, or may be present in underlying signals from which the nociception parameters are derived. For example, such noise may cause the nociception monitoring system to sense increases in the nociception parameters of the patient above the nociception threshold even when there is not a corresponding increase in the surgical stress experienced by the patient. If the clinician were to increase the amount of analgesic administered to the patient in response to such false positive indications of a severe nociceptive stimulus, then the clinician may unwittingly administer additional analgesic to the patient where it may not be required. In addition, different patients may respond differently to surgical stress and stimuli, such that the same level of nociception parameters of different patients may indicate different levels of surgical stress experienced by different patients. These different responses may be due to the physiology of patients, the amount of analgesic already administered to the patients, and the like.

In addition, if the nociception parameter repeatedly crosses the nociception threshold in a short amount of time, such that the nociception parameter increases to reach or exceed the nociception parameter and then decreases to drop below the nociception parameter multiple times in the short amount of time, it may be difficult for the clinician to determine when or whether to administer additional analgesic to the patient. Further, it may be impracticable for the clinician to increase and subsequently reduce the amount of analgesic administered to the patient multiple times as the nociception parameters rises above and dips below the nociception parameter multiple times throughout the short amount of time.

This disclosure describes devices, systems, and methods for determining a characteristic nociception parameter based on the nociception parameter of the patient, where the characteristic nociception parameter can be compared with the nociception threshold of a patient to control analgesic delivery to the patient. The control can include, for example, determining whether and/or when to deliver or increase an amount of analgesic administered to the patient. Aspects of this disclosure describe techniques with which a nociception monitoring system monitors the nociception parameter of the patient and to determine, at a point in time, the characteristic nociception parameter at the point in time based at least in part on values of the nociception parameter over a period of time. For example, the nociception monitoring system may determine a characteristic nociception parameter as an average of the values of the nociception parameter over the period of time, as a weighted average of the values of the nociception parameter over the period of time, and the like.

The nociception monitoring system may compare the characteristic nociception parameter determined at the point in time with a nociception threshold to determine whether the patient is experiencing a severe nociceptive stimulus (e.g., referred to herein as a nociception event) at the point in time. For example, if the characteristic nociception parameter is greater than or equal to the nociception threshold, then the nociception monitoring system may determine that a nociception event has occurred at the point in time. In some examples, if the nociception monitoring system determines that a nociception event has occurred at the point in time, then the nociception monitoring system may generate an output that is indicative of the detected nociception event. The output may, for example, indicate that a clinician should increase the amount of analgesic administered to the patient or provide another suitable indication to the clinician related to the delivery of the analgesic or another action the clinician may take.

By determining a characteristic nociception parameter based on the values of the nociception parameter over a period of time, the devices, systems, and techniques of this disclosure may more clearly determine the points in time a clinician should adjust the amount of analgesic administered to the patient to dampen the surgical stress experienced by the patient as indicated by the nociception parameter of the patient. The techniques of this disclosure therefore enables a clinician or an analgesic administration system to more accurately and timely administer analgesic to the patient when it may be required to reduce the surgical stress caused to the patient and to decrease unnecessary administration of additional analgesic administered to the patient due to false positives.

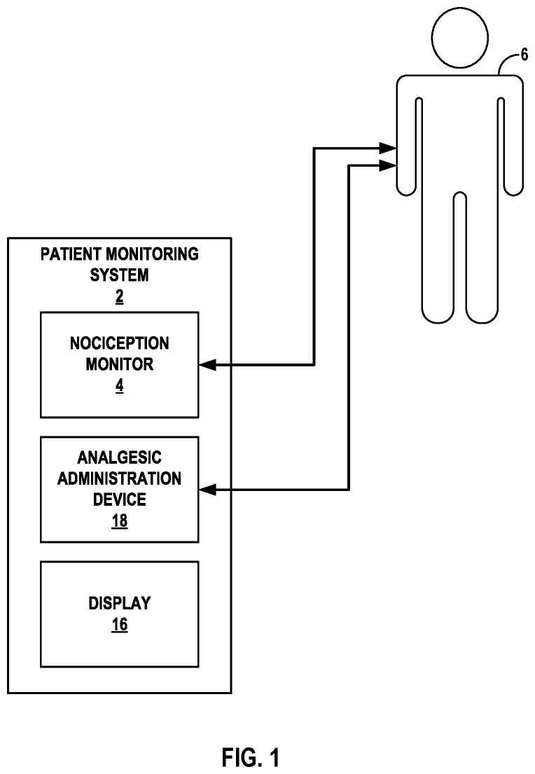

FIG. 1 is a block diagram illustrating an example environment in which a patient monitoring system monitors one or more nociception parameters of a patient undergoing a medical procedure. As shown in FIG. 1, patient monitoring system 2 may monitor one or more physiological signals of patient 6 to determine the amount of surgical stress experienced by patient 6 during the medical procedure. By monitoring the amount of surgical stress experienced by patient 6, patient monitoring system 2 or a clinician that uses patient monitoring system 2 may be able to determine whether to adjust (e.g., increase or decrease a quantity or adjust a timing) an amount of analgesic administered to patient 6 during the medical procedure.

In some examples, patient monitoring system 2 is configured to monitor patient 6 during a medical procedure, such as surgery, and configured to titrate analgesic or anesthetic delivered to patient 6 during surgery to provide anesthesia for patient 6. For example, patient monitoring system 2 may titrate the analgesic or anesthetic automatically, without significant or any clinician intervention, or based on manual inputs by a clinician. Patient monitoring system 2 may include nociception monitor 4, analgesic administration device 18, and display 16. As a medical procedure is performed on patient 6, nociception monitor 4 of patient monitoring system 2 may monitor the amount of surgical stress experienced by patient 6 by monitoring one or more physiological signals of patient 6, such as, but not limited to one or more of an ECG, a PPG, an EEG, the skin conductance of patient 6, the body temperature of patient 6, a respiratory rate, and the like, to determine a measure of a nociception parameter associated with patient 6 during the surgery, where the nociception parameter corresponds to the amount of surgical stress experienced by patient 6. In some examples, the nociception parameter may be an integer, and may range from, for example, 0 to 100. As such, by determining a continuous measure of a nociception parameter associated with patient 6 during the surgery, nociception monitor 4 may determine a continuous measure of the amount of surgical stress experienced by patient 6 during surgery.

In some examples, system 2 continuously determines the measure of the nociception parameter. In other examples, system 2 periodically determines the measure of the nociception parameter.

Display 16 is configured to display the nociception parameter over time. For example, as nociception monitor 4 determines the nociception parameter associated with patient 6, display 16 may output a graphical representation of the nociception parameter over time, which may be viewed by a clinician to monitor the amount of surgical stress experienced by patient 6.

In some examples, patient monitoring system 2 includes analgesic administration device 18, which may include one or more components and/or devices configured to administer analgesic to patient 6 during surgery. Analgesic administration device 18 may be coupled to patient 6, such as via one or more intravenous (IV) lines, a breathing mask, a tube, and the like, in order to provide analgesia to patient 6 during surgery.

In some examples, the analgesic administration device 18 is configured to administer analgesic to patient 6 without user intervention from, for example, a clinician. That is, patient monitoring system 2 may control the amount of analgesic being administered by analgesic administration device 18 to patient 6 (i.e., automatically titrate analgesic delivered to patient 6), such as by increasing the amount of analgesic administered by analgesic administration device 18 to patient 6 and/or by decreasing the amount of analgesic administered by analgesic administration device 18 to patient 6, without user intervention.

In addition to or instead of an automatic administration of analgesia by analgesic administration device 18, in some examples, a clinician may control the amount of analgesic being administered by analgesic administration device 18 to patient 6. For example, the clinician may provide user input to patient monitoring system 2 indicative of the amount of analgesic to be administered by analgesic administration device 18 to patient 6. Patient monitoring system 2 may receive such user input indicative of the amount of analgesic being administered by analgesic administration device 18 to patient 6 and may, in response, control analgesic administration device 18 to administer the amount of analgesic to patient 6 indicated by the user input.

As a medical procedure is performed on patient 6, nociception monitor 4 of patient monitoring system 2 may continuously or periodically determine the nociception parameter associated with patient 6 in order to monitor the amount of surgical stress experienced by patient 6. In some examples, nociception monitor 4 may specify a nociception threshold for patient 6, where nociception parameters of patient 6 that are at or above the nociception threshold may be indicative of patient 6 experiencing a severe nociceptive stimulus (referred to herein as a nociception event). In other examples, if the nociception parameter of patient 6 is at or below the nociception threshold, the nociception parameter may be indicative of patient 6 experiencing a severe nociceptive stimulus. In the example in which the nociception parameter of patient 6 is in a numerical range from 0 to 100, a nociception threshold may also be an integer value in a numerical range, e.g., between 0 and 100, such as 70, 80, and the like. The nociception threshold may be preset and may be the same for all patients, or may be adjusted, e.g., by a clinician, to a value that may be different for different patient.

As such, in some examples, if nociception monitor 4 determines that the nociception parameter of patient 6 is greater than or equal to the nociception threshold, then processing circuitry (not shown) of patient monitoring system 2 may detect a nociception event and may accordingly cause analgesic administration device 18 to increase the amount of analgesic administered to patient 6 to dampen down the surgical stress experienced by patient 6 and to decrease the nociception parameter of patient 6 to below the nociception threshold. In other examples, if nociception monitor 4 determines that the nociception parameter of patient 6 is greater than or equal to the nociception threshold, then processing circuitry of patient monitoring system 2 may provide an indication via display 16 or another user output device (e.g., audio circuitry configured to generate an audible output or circuitry configured to generate a tactile output perceived by a clinician) to indicate to adjust an amount of analgesic administered to patient 6.

In some examples, a clinician may determine a patient-specific nociception threshold based on making a tradeoff between the amount of surgical stress endured by patient 6 and the amount of analgesic administered to patient 6. For example, setting a higher nociception threshold may lead to relatively less analgesic being administered to patient 6, thereby leading patient 6 to endure relatively more surgical stress. On the other hand, setting a lower nociception threshold may lead to relatively more analgesic being administered to patient 6, thereby leading patient 6 to endure relatively less surgical stress.

In some examples, setting the nociception threshold too high may lead to underdosing patient 6 with analgesia, thereby causing the patient 6 to experience too much surgical stress, which may lead to poorer outcomes. In some examples, setting the nociception threshold too low may lead to delivery of unnecessary doses of analgesic to patient 6, which may lead to patient 6 developing hyperalgesia after the medical procedure. Thus, if patient 6 is more susceptible to hyperalgesia, then the clinician may choose to set a relatively higher nociception threshold for patient 6 to decrease the possibility of patient 6 developing hyperalgesia after the medical procedure.

In some examples, a clinician may determine an upper nociception threshold and a lower nociception threshold for patient 6, where the nociception parameter being greater than or equal to the upper nociception threshold may be indicative of underdosing patient 6 with analgesia, and where the nociception parameter being less than or equal to the lower nociception threshold may be indicative of delivering more analgesic than necessary to patient 6. In these examples, when processing circuitry of patient monitoring system 2 determines that the nociception parameter is greater than or equal to the upper nociception threshold, the processing circuitry may detect a nociception event and may accordingly cause analgesic administration device 18 to increase the amount of analgesic administered to patient 6 to dampen down the surgical stress experienced by patient 6 and to decrease the nociception parameter of patient 6 to below the upper nociception threshold. Conversely, when processing circuitry of patient monitoring system 2 determines that the nociception parameter is less than or equal to the lower nociception threshold, the processing circuitry may detect a nociception event and may accordingly cause analgesic administration device 18 to decrease the amount of analgesic administered to patient 6, thereby possibly causing the nociception parameter of patient 6 to increase above the lower nociception threshold.

In some examples, instead of comparing the nociception parameter of patient 6 with the nociception threshold to determine whether a nociception event has occurred, processing circuitry of patient monitoring system 2 may determine a characteristic nociception parameter based at least in part on a plurality of nociception parameters of patient 6 sensed over a period of time. Processing circuitry of patient monitoring system 2 may compare the characteristic nociception parameter with the nociception threshold to determine whether a nociception event has occurred. For example, if the characteristic nociception parameter is greater than or equal to the nociception threshold, processing circuitry of patient monitoring system 2 may determine that a nociception event has occurred. In other examples, if the characteristic nociception parameter is less than or equal to the nociception threshold, processing circuitry of patient monitoring system 2 may determine that a nociception event has occurred.

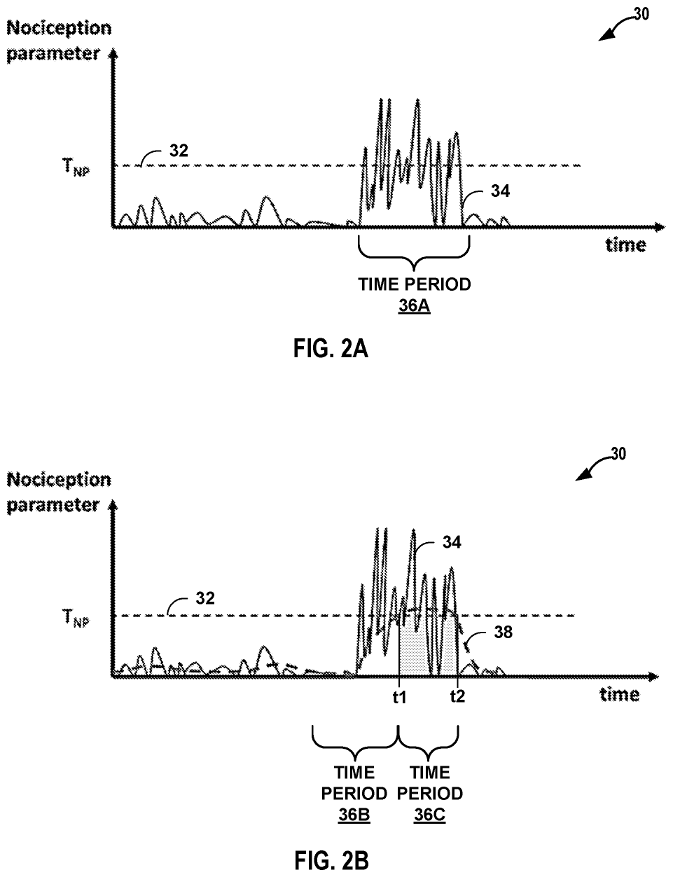

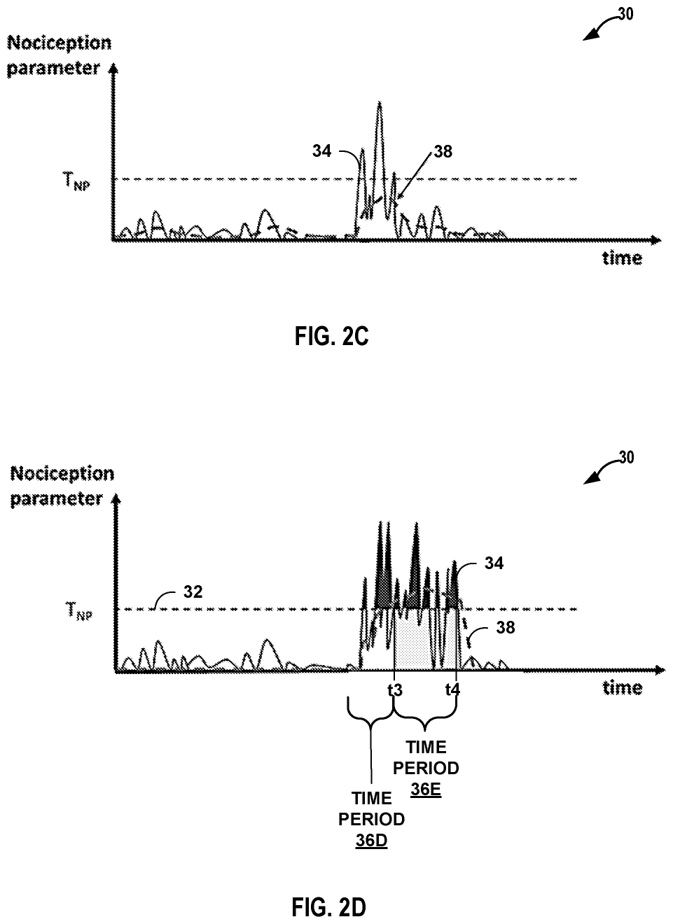

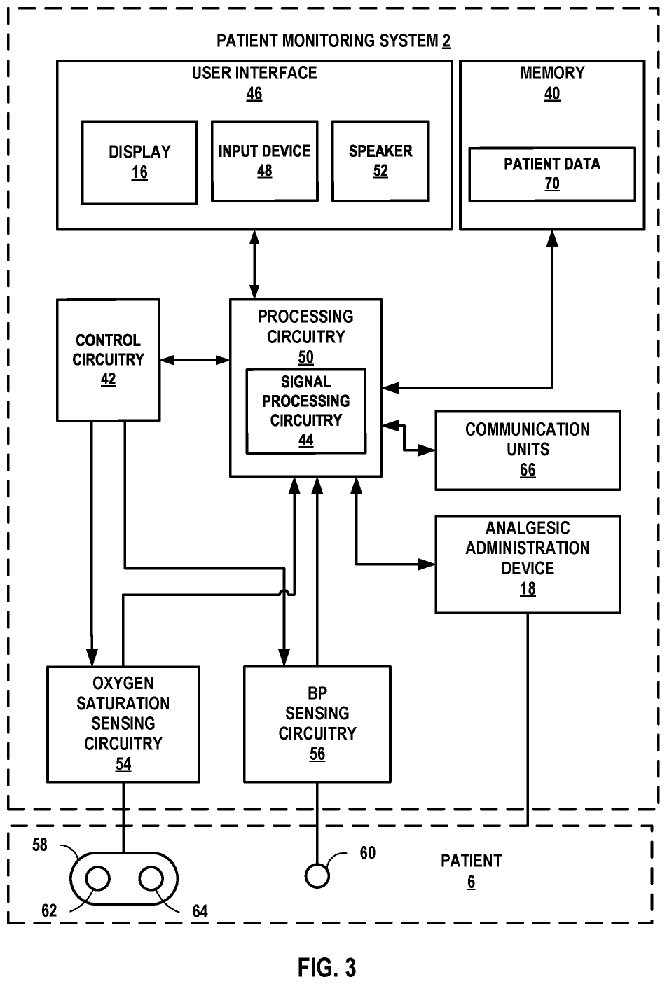

FIGS. 2A-2D illustrate example techniques for determining a characteristic nociception parameter, in accordance with aspects of this disclosure. At a point in time when nociception monitor 4 monitors the nociception parameter of patient 6, processing circuitry 50 (FIG. 3) of patient monitoring system 2 may determine a characteristic nociception parameter at the point in time based at least in part on the nociception parameter of patient 6. In some examples, the point in time may be an instant in time or may be a time period. Processing circuitry 50 may compare the characteristic nociception parameter with the nociception threshold to determine whether a nociception event has occurred at the point in time. If processing circuitry 50 determines that a nociception event has occurred at the point in time, then processing circuitry 50 may provide an indication (e.g., via display 16 or another user output device) to indicate to a clinician to adjust (e.g., increase) an amount of analgesic administered to patient 6, or may cause analgesic administration device 18 to increase the amount of analgesic administered to patient 6 to dampen down the surgical stress indicated by the nociception event.

As shown in FIG. 2A, time graph 30 is a visual representation of the nociception parameter 34 of patient 6 over time during a medical procedure, such as monitored by nociception monitor 4 compared with nociception threshold 32. Nociception threshold 32 can be, for example, a predetermined nociception parameter value that is stored by a memory of patient monitoring system 2 or a memory of another device, and can be specific to patient 6 or more general and used for a plurality of patients. As shown in FIG. 2A, nociception parameter 34 oscillates to repeatedly rise to be equal to or above nociception threshold 32 and to repeatedly dip to be below nociception threshold 32.

If time period 36A is relatively short (e.g., five seconds to ten seconds in length), the number of times nociception parameter 34 rises from being below nociception threshold 32 to being greater than or equal to nociception threshold 32 may make it difficult for a clinician to determine time points within time period 36A at which to increase the amount of analgesic administered to patient 6. For example, if time nociception parameter 34 remains at or above nociception threshold 32 within time period 36A for less than a second each time nociception parameter 34 rises from being below nociception threshold 32 to being greater than or equal to nociception threshold 32, a clinician may not be able to react quickly enough to increase the amount of analgesic administered to patient 6 before nociception parameter 34 decreases back below nociception threshold 32.

To more clearly identify when to increase the amount of analgesic administered to patient 6, instead of simply comparing the nociception parameter 34 to nociception threshold 32, processing circuitry 50 of patient monitoring system 2 may derive a characteristic nociception parameter from nociception parameter 34 that more better indicates a nociception event for which an adjustment to analgesic delivered to patient 6 may be desirable. The nociception event may better indicate the one or more points in time when the amount of analgesic administered to patient 6 should be adjusted (e.g., increased). In some examples, processing circuitry 50 may determine a characteristic nociception parameter at a point in time based at least in part on values of nociception parameter 34 over a period of time prior to the respective point in time.

In some examples, the period of time (also referred to as a “time period”) may have a duration of 30 seconds, 60 seconds, 120 seconds, 180 seconds, 240 seconds, and the like, and may occur prior to the point in time at which processing circuitry 50 determines the characteristic nociception parameter. The values of nociception parameter 34 during the time period may be the values of nociception parameter 34 sensed and obtained by nociception monitor 4 during the time period. For example, if nociception monitor 4 senses and obtains the value of nociception parameter 34 of patient 6 once every second, then, for a time period having a duration of 30 seconds, the values of nociception parameter 34 during the time period of 30 seconds may include 30 values of nociception parameter 34, each of which corresponds to a second of the 30 second time period. In other examples, nociception monitor 4 may sense and obtain the value of nociception parameter 34 of patient 6 once every half a second, once every 2 seconds, once every 5 seconds, and the like.

In some examples, to determine a characteristic nociception parameter at a point in time based at least in part on values of nociception parameter 34 over a period of time, processing circuitry 50 may determine a characteristic nociception parameter at a point in time as an average (e.g., arithmetic mean) of the values of nociception parameter 34 over a period of time. The characteristic nociception parameter can be expressed as follows:

$$N P {C}_{i} = \frac{{\sum }_{j = i - N + 1}^{i} N {P}_{j}}{N} ,$$

where NPCᵢ is the characteristic nociception parameter at current time index i, that corresponds to a point in time, NPS is the value of nociception parameter 34 at time index j, and N is the number of samples (i.e., values of nociception parameter 34) obtained during the time period.

In some examples, to determine a characteristic nociception parameter at a point in time based at least in part on values of nociception parameter 34 over a period of time, processing circuitry 50 may determine a characteristic nociception parameter at a point in time as a weighted average (e.g., weighted arithmetic mean) of the values of nociception parameter 34 over a period of time. That is, processing circuitry 50 may place a relatively higher weight on one or more values of nociception parameter 34 during the period of time, such as by at least multiplying each of the one or more values of nociception parameter 34 with a relatively high value, and may place a relatively lower weight on one or more other values of nociception parameter 34 during the period of time such as by at least multiplying each of the one or more other values of nociception parameter 34 with a relatively low value or by at least not multiplying the values by the high value associated with the higher weight. In other examples, processing circuitry 50 may place a relatively lower weight on one or values of nociception parameter 34 during the period of time by at least multiplying each of the one or more other values of nociception parameter 34 with a relatively low value and may place a relatively higher weight on one or more values of nociception parameter 34 during the period of time by at least not multiplying the values by any value.

In some examples, the weight applied to a nociception parameter value is based on a recency of the value relatively to the current time. For example, processing circuitry 50 may place a relatively higher weight on relatively more recent values of nociception parameter 34 during the period of time and/or may place a relatively lower weight on other values of nociception parameter 34 during the period of time. In some examples, processing circuitry 50 may weigh the values of nociception parameter 34 during the period of time based on internal noise metrics. For example, processing circuitry of patient monitoring system 2 may place a relatively higher weight on one or more values of nociception parameter 34 during the period of time that correspond to nociception parameter 34 signals being associated a low noise metric and may place a relatively lesser weight on one or more values of nociception parameter 34 during the period of time that correspond to nociception parameter 34 signals being associated a high noise.

A noise metric may be derived from constituent signals that make up nociception parameter 34. For example, if nociception parameter 34 is derived from a PPG signal, an oximeter used to obtain the PPG signal may provide or signal noise flags that correspond to, e.g., motion detected by the oximeter. In this example, nociception parameter 34 derived from a PPG signal during which oximeter output a noise flag (e.g., when the oximeter detects motion) may be considered to be associated with a high noise metric, while nociception parameter 34 derived from a PPG signal during which oximeter does not output a noise flag (e.g., when the oximeter does not detect motion) may be considered to be associated with a high noise metric.

In some examples, a noise metric may be derived from nociception parameter 34 itself. For example, the amount of dropouts in the nociception parameter 34 during any given time period can be tracked. In this example, nociception parameter 34 during time periods having a higher amount of dropouts of the nociception parameter 34 may be associated with a higher noise metric, while nociception parameter 34 during time periods having a lower amount of dropouts of the nociception parameter 34 may be associated with a lower noise metric. In some examples, a noise metric may be derived from variations in nociception parameter 34 during any given time period, where nociception parameter 34 during time periods having a higher variation in nociception parameter 34 may be associated with a higher noise metric, while nociception parameter 34 during time periods having a lower variation in nociception parameter 34 may be associated with a lower noise metric.

In some examples, to determine a characteristic nociception parameter at a point in time based at least in part on values of nociception parameter 34 over a period of time, processing circuitry 50 may use an infinite impulse response (IIR) filter to determine a characteristic nociception parameter at a point in time as a weighted combination of a previous characteristic nociception parameter determined at a previous point in time summed with a new nociception parameter signal value derived from the value of nociception parameter 34 at the point in time. For example, the characteristic nociception parameter can be expressed as follows:

NPCᵢ=w*NPCᵢ₋₁+(1−w)*NPᵢ,

where NPCᵢ is the characteristic nociception parameter at current time index i, that corresponds to a point in time, where NPCᵢ₋₁ is the most recent previous characteristic nociception parameter determined based on nociception parameter 34, where NPᵢ is the value of nociception parameter 34 at the current time index i, that corresponds to the point in time, and where w is a weight between 0 and 1.

In this example, processing circuitry 50 may determine the characteristic nociception parameter at a point in time based on the most recently determined characteristic nociception parameter prior to the point in time and the value nociception parameter 34 at the point in time, where the most recently determined characteristic nociception parameter prior to the point in time may have been determined via any of the techniques described herein. Processing circuitry 50 may determine the characteristic nociception parameter at the point in time by at least multiplying a weight w to the most recently determined characteristic nociception parameter and may sum the result with the result of multiplying one minus the weight w with the value nociception parameter 34 at the point in time.

As shown in FIG. 2B, as nociception monitor 4 monitors nociception parameter 34 of patient 6, processing circuitry 50 may continuously determine characteristic nociception parameter 38 based at least in part on nociception parameter 34. For example, processing circuitry 50 may periodically, such as every second, every 2 seconds, ever 10 seconds, and the like, determine the value of characteristic nociception parameter 38.

For example, processing circuitry 50 may determine characteristic nociception parameter 38 at a point in time, such as at time t1, to determine whether a nociception event has occurred at the point in time. Processing circuitry 50 may determine characteristic nociception parameter 38 at time t1 based at least in part on values of the nociception parameter 34 over a period of time that at least partially precedes time t1. That is, in some examples, the period of time may totally precede time t1, while in other examples the period of time may include time t1 at which characteristic nociception parameter 38 is determined based on the values of nociception parameter 34 during the time period.

In the example of FIG. 2B, processing circuitry 50 may determine characteristic nociception parameter 38 at time t1 based at least in part on values of nociception parameter 34 over time period 36B that at least partially precedes time t1. In some examples, time period 36B may completely precede time t1, such as by immediately preceding t1. In some examples, time period 36B may include time t1. In any event, time period 36B that processing circuitry 50 may use for determining characteristic nociception parameter 38 at time t1 may not include any time after time t1.

The values of nociception parameter 34 during time period 36B may include values of nociception parameter 34 obtained by nociception monitor 4 during time period 36B while monitoring nociception parameter 34 of patient 6. As described above, nociception monitor 4 may monitor nociception parameter 34 of patient 6 to periodically obtain values of nociception parameter 34, such as every second, every 5 seconds, and the like.

In some examples, processing circuitry 50 may determine characteristic nociception parameter 38 at time t1 as the average (e.g., arithmetic mean) of the values of nociception parameter 34 obtained by nociception monitor 4 during time period 36B. In some examples, processing circuitry 50 may determine characteristic nociception parameter 38 at time t1 as the weighted average (e.g., weighted arithmetic mean) of the values of nociception parameter 34 obtained by nociception monitor 4 during time period 36B.

Processing circuitry 50 may, for example, weigh the values of nociception parameter 34 over time period 36B based on recency, such that processing circuitry of patient monitoring system 2 may place greater weight on the more recent values of nociception parameter 34 in time period 36B and may place lesser weight on the less recent values of nociception parameter 34 in time period 36B. In another example, processing circuitry 50 may weigh the values of nociception parameter 34 over time period 36B based on one or more noise metrics, such that processing circuitry of patient monitoring system 2 may place greater weight on values of nociception parameter 34 in time period 36B associated with less noise and may place lesser weight on the values of nociception parameter 34 in time period 36B associated with greater noise.

In some examples, processing circuitry 50 may determine characteristic nociception parameter 38 at time t1 based at least in part on a previously determined characteristic nociception parameter 38. For example, processing circuitry 50 may determine characteristic nociception parameter 38 at time t1 based on a most recently determined characteristic nociception parameter 38 and the value of nociception parameter 34 at time t1, such as by multiplying the most recently determined characteristic nociception parameter 38 with a weight w, multiplying the value of nociception parameter 34 at time t1 with one minus w, and summing the result as the characteristic nociception parameter 38 at time t1.

In the example of FIG. 2B, characteristic nociception parameter 38 is greater than nociception threshold 32. As such, processing circuitry 50 may determine that a nociception event has occurred at time t1. As nociception monitor 4 continues to monitor the nociception parameter 34 of patient 6 after time t1, processing circuitry 50 may continue to periodically determine, over time, the characteristic nociception parameter 38 of patient 6. As can be seen in FIG. 2B, characteristic nociception parameter 38 may be greater than or equal to nociception threshold 32 in time period 36C from time t1 until time t2. Thus, processing circuitry 50 may determine that a nociception event has occurred that spans time period 36C, and may, during time period 36C, output one or more indications of the occurrence of a nociception event that spans time period 36C.

As shown in FIG. 2C, if characteristic nociception parameter 38 never reaches nociception threshold 32, then processing circuitry 50 may determine that no nociception events have occurred even if nociception parameter 34 is greater than or equal to nociception threshold 32. In this case, processing circuitry 50 may not output any indications of the occurrence of a nociception event.

In some examples, processing circuitry 50 may determine a characteristic nociception parameter 38 based at least in part on integrating (i.e., determining an integral of) the values of the nociception parameter 34 that is greater than or equal to nociception threshold 32 over a period of time. As shown in FIG. 2D, processing circuitry 50 may determine characteristic nociception parameter 38 at time t3 based on integrating the values of nociception parameter 34 within time period 36D that are greater than or equal to nociception threshold 32, which are represented in FIG. 2D as dark shaded areas, and dividing the integral by time period 36D over which the values were integrated.

In the example of FIG. 2D, characteristic nociception parameter 38 is greater than nociception threshold 32 at time t3. As such, processing circuitry 50 may determine that a nociception event has occurred at time t3. As nociception monitor 4 continues to monitor the nociception parameter 34 of patient 6 after time t3, processing circuitry 50 may continue to periodically determine, over time, the characteristic nociception parameter 38 of patient 6. As can be seen in FIG. 2D, characteristic nociception parameter 38 may be greater than or equal to nociception threshold 32 in time period 36E from time t3 until time t4. Thus, processing circuitry 50 may determine that a nociception event has occurred that spans time period 36E, and may, during time period 36E, output one or more indications of the occurrence of a nociception event that spans time period 36E.

Processing circuitry 50 may, in response to determining an occurrence of a nociception event for patient 6, provide an indication of the nociception event, such as by generating and presenting an alert via display 16 or another output device including output circuitry. In some examples, processing circuitry 50 may, in response to determining an occurrence of a nociception event for patient 6, provide an indication to adjust an amount of analgesic to administer to patient 6 via display or another output device including output circuitry. Thus, in these examples, if processing circuitry 50 determines that the characteristic nociception parameter 38 is greater than or equal to the nociception threshold 32, then processing circuitry of patient monitoring system 2 may provide an indication to adjust an amount of analgesic to administer to patient 6, such as by providing an indication to increase the amount of analgesic to administer to patient 6.

In some examples, a clinician may manually control analgesic administration device 18 to administer analgesic to patient 6. As such, in order to provide an indication to adjust an amount of analgesic to administer to patient 6, processing circuitry 50 may output, for display at display 16, an indication to a clinician to adjust the amount of analgesic administered to patient 6. For example, processing circuitry 50 may output, for display at display 16, an indication of the amount of analgesic to administer to patient 6 or a more general instruction or suggestion to the clinician to increase or otherwise adjust the amount of analgesic.

In some examples, processing circuitry 50 may be able to control analgesic administration device 18 to administer analgesic to patient 6 without user intervention. As such, in order to provide an indication to adjust an amount of analgesic to administer to patient 6, processing circuitry 50 may output a signal to analgesic administration device 18 to direct analgesic administration device 18 to increase or otherwise adjust the amount of analgesic to administer to patient 6. Analgesic administration device 18 may, in response to receiving the signal, increase or otherwise adjust the amount of analgesic to administer to patient 6.

In some examples, processing circuitry 50 may determine how much to adjust the amount of analgesic administered to patient 6 and/or whether to adjust the amount of analgesic administered to patient 6 based on the current level of analgesic administered to patient 6 and/or the total amount of analgesic administered to patient 6 during the current medical procedure. In some examples, processing circuitry 50 may limit the amount of analgesic administered to patient 6 at any point in time to a specified analgesic level. As such, processing circuitry 50 may increase the amount of analgesic administered to patient 6 at a point in time to no more than the specified analgesic level. If processing circuitry 50 determines that increasing the amount of analgesic administered to patient 6 would cause the amount of analgesic administered to patient 6 to rise above the specified analgesic level, then processing circuitry 50 may refrain from increasing the amount of analgesic administered to patient 6 or providing an instruction to increase the amount of analgesic via display 16.

In some examples, processing circuitry 50 may determine how much to adjust the amount of analgesic administered to patient 6 and/or whether to adjust the amount of analgesic administered to patient 6 based on the total amount of analgesic administered to patient 6 during the course of the surgery or other medical procedure. For example, the total amount of analgesic administered to patient 6 over the course of the surgery may not exceed a total analgesic limit. If processing circuitry 50 determines that increasing the amount of analgesic administered to patient 6 would cause the total amount of analgesic administered to patient 6 over the course of the surgery to rise above the total analgesic limit, then processing circuitry 50 may refrain from increasing the amount of analgesic administered to patient 6 providing an instruction to increase the amount of analgesic via display 16. Processing circuitry 50 may determine how much to increase the amount of analgesic administered to patient 6 using any techniques described above alone or in combination with each other.

The techniques described herein may provide one or more advantages. By determining whether a nociception event has occurred based on determining a characteristic nociception value based on the values of the nociception parameter over a period of time, rather than based on a single nociception parameter value at one specific point in time, patient monitoring system 2 may be able to more accurately determine when a nociception event has occurred. Being able to more accurately detect the occurrence of nociception events may enable patient monitoring system 2 to better administer (e.g., more timely) the proper amount of analgesic to patient 6. The proper amount of analgesic can be, for example, an amount of analgesic necessary to provide the desired analgesia outcomes for patient 6 but not being too much analgesic, which may lead to undesirable outcomes for patient 6. Administering a more proper amount of analgesic to the patient (e.g., better corresponding to surgical stress experienced by patient 6 during surgery using the techniques described herein may have one or more beneficial outcomes, such as leading to reductions in opioid administration during and after surgery, post-operative pain scores, the length of the hospital stay, and/or post-operative complications.

FIG. 3 is a block diagram illustrating an example of the patient monitoring system 2 of FIG. 1. As shown in FIG. 3, in some examples, patient monitoring system 2 includes analgesic administration device 18, memory 40, control circuitry 42, user interface 46, processing circuitry 50, sensing circuitry 54 and 56, sensing devices 58 and 60, and one or more communication units 66. In the example shown in FIG. 3, user interface 46 includes display 16, input device 48, and/or speaker 52, which may be any suitable audio device including circuitry configured to generate and output a sound and/or noise. In some examples, patient monitoring system 2 may be configured to determine and output (e.g., for display at display 16) the nociception parameter of a patient 6 during a medical procedure.

Processing circuitry 50, as well as other processors, processing circuitry, controllers, control circuitry, and the like, described herein, may include one or more processors. Processing circuitry 50 and control circuitry 42 may each include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 50 and/or control circuitry 42 may include multiple components, such as any combination of one or more microprocessors, one or more DSPs, one or more ASICs, or one or more FPGAs, as well as other discrete or integrated logic circuitry, and/or analog circuitry.

Control circuitry 42 may be operatively coupled to processing circuitry 50. Control circuitry 42 is configured to control an operation of sensing devices 58 and 60. In some examples, control circuitry 42 may be configured to provide timing control signals to coordinate operation of sensing devices 58 and 60. For example, sensing circuitry 54 and 56 may receive from control circuitry 42 one or more timing control signals, which may be used by sensing circuitry 54 and 56 to turn on and off respective sensing devices 58 and 60, such as to periodically collect calibration data using sensing devices 58 and 60. In some examples, processing circuitry 50 may use the timing control signals to operate synchronously with sensing circuitry 54 and 56. For example, processing circuitry 50 may synchronize the operation of an analog-to-digital converter and a demultiplexer with sensing circuitry 54 and 56 based on the timing control signals.

One or more communication units 66 include circuitry operable to communicate with one or more devices external to patient monitoring system 2 via one or more networks by transmitting and/or receiving network signals on the one or more networks such as the Internet, a Wide Area Network, a Local Area Network, and the like. Examples of one or more communication units 66 include a network interface card (e.g. such as an Ethernet card), an optical transceiver, a radio frequency transceiver, or any other type of device that can send and/or receive information. Other examples of one or more communication units 66 may include Near-Field Communications (NFC) units, Bluetooth® radios, short wave radios, cellular data radios, wireless network (e.g., Wi-Fi®) radios, as well as universal serial bus (USB) controllers.

Memory 40 may be configured to store, for example, patient data 70. For example, processing circuitry 50 may store various data associated with patient 6 in patient data 70. For example, processing circuitry 50 may store the nociception parameter of patient 6, a nociception threshold (e.g., including upper and lower threshold values in some examples), one or more determined characteristic nociception parameters, a current total amount of analgesic administered to patient 6, a current level of analgesic being administered to patient 6, and the like in patient data 70 in memory 40. The nociception threshold can be specific to patient 6 or used for a population of patients.

In some examples, memory 40 may store program instructions. The program instructions may include one or more program modules that are executable by processing circuitry 50. When executed by processing circuitry 50, such program instructions may cause processing circuitry 50 to provide the functionality ascribed to it herein. The program instructions may be embodied in software, firmware, and/or RAMware. Memory 40 may include any one or more of volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

User interface 46 may include a display 16, an input device 48, and a speaker 52. In some examples, user interface 46 may include fewer or additional components. User interface 46 is configured to present information to a user (e.g., a clinician). For example, user interface 46 and/or display 16 may include a monitor, cathode ray tube display, a flat panel display such as a liquid crystal (LCD) display, a plasma display, a light emitting diode (LED) display, and/or any other suitable display. In some examples, user interface 46 may be part of a multiparameter monitor (VIPM) or other physiological signal monitor used in a clinical or other setting, a personal digital assistant, mobile phone, tablet computer, laptop computer, any other suitable computing device, or any combination thereof, with a built-in display or a separate display.

In some examples, processing circuitry 50 may be configured to present, by user interface 46, such as display 16, a graphical user interface to a user. The graphical user interface can include information regarding the delivery of analgesic or anesthesia to patient 6, one or more sensed nociception parameters, one or more characteristic nociception parameters, and the like. For example, the graphical user interface may include one or more of time graphs 30 of FIGS. 2A-2D of the nociception parameter of patient 6 over time, one or more characteristic nociception parameters shown relatively to the time graphs, and indications of occurrences of nociception events. In some examples, the graphical user interface can also include an instruction or suggestion to a clinician to administer additional analgesics or anesthesia or otherwise adjust the delivery of analgesics, anesthesia, or other pharmaceutical agents or fluids. User interface 46 may also include means for projecting audio to a user, such as speaker 52.

In some examples, processing circuitry 50 may also receive input signals from additional sources (not shown), such as a user. For example, processing circuitry 50 may receive from input device 48, such as a keyboard, a mouse, a touch screen, buttons, switches, a microphone, a joystick, a touch pad, or any other suitable input device or combination of input devices, an input signal. The input signal may contain information about patient 6, such as physiological parameters, treatments provided to patient 6, or the like. Additional input signals may be used by processing circuitry 50 in any of the determinations or operations it performs in accordance with examples described herein. For example, the input processing circuitry 50 receives via input device 48 can indicate the occurrence of a medical event, based on which processing circuitry 50 may determine a patient-specific nociception threshold.

In some examples, processing circuitry 50 and user interface 46 may be part of the same device or supported within one housing (e.g., a computer or monitor). In other examples, processing circuitry 50 and user interface 46 may be separate devices configured to communicate through a wired connection or a wireless connection.

Sensing circuitry 54 and 56 is configured to receive signals (“physiological signals”) indicative of physiological parameters from respective sensing devices 58 and 60 and communicate the physiological signals to processing circuitry 50. Sensing devices 58 and may include any sensing hardware configured to sense a physiological parameter of a patient, e.g., indicative of a nociception response of patient 6. Example sensing hardware includes, but is not limited to, one or more electrodes, light sources, optical receivers, blood pressure cuffs, or the like. The sensed physiological signals may include signals indicative of physiological parameters from a patient, such as, but not limited to, blood pressure, blood oxygen saturation (e.g., pulse oximetry and/or regional oxygen saturation), blood volume, heart rate, heart rate variability, skin conductance, and respiration. For example, sensing circuitry 54 and 56 may include, but are not limited to, blood pressure sensing circuitry, blood oxygen saturation sensing circuitry, blood volume sensing circuitry, heart rate sensing circuitry, temperature sensing circuitry, electrocardiography (ECG) sensing circuitry, electroencephalogram (EEG) sensing circuitry, electromyogram (EMG) sensing circuitry or any combination thereof.

In some examples, sensing circuitry 54 and 56 and/or processing circuitry 50 may include signal processing circuitry 44 configured to perform any suitable analog conditioning of the sensed physiological signals. For example, sensing circuitry 54 and 56 may communicate to processing circuitry 50 an unaltered (e.g., raw) signal. Processing circuitry 50, e.g., signal processing circuitry 44, may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

In some examples, the conditioned analog signals may be processed by an analog-to-digital converter of signal processing circuitry 44 to convert the conditioned analog signals into digital signals. In some examples, signal processing circuitry 44 may operate on the analog or digital form of the signals to separate out different components of the signals. In some examples, signal processing circuitry 44 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. In some examples, signal processing circuitry 44 may decrease the number of samples in the digital detector signals. In some examples, signal processing circuitry 44 may remove dark or ambient contributions to the received signal. Additionally, or alternatively, sensing circuitry 54 and 56 may include signal processing circuitry 44 to modify one or more raw signals and communicate to processing circuitry 50 one or more modified signals.

In the example shown in FIG. 3, patient monitoring system 2 includes an oxygen saturation sensing device 58 (also referred to herein as blood oxygen saturation sensing device 58), which is configured to generate an oxygen saturation signal indicative of blood oxygen saturation within the venous, arterial, and/or capillary systems within a region of patient 6. For example, oxygen saturation sensing device 58 may include a sensor configured to non-invasively generate a plethysmography (PPG) signal. One example of such a sensor may be one or more oximetry sensors (e.g., one or more pulse oximetry sensors) placed at one or multiple locations on patient 6, such as at a fingertip of patient 6, an earlobe of patient 6, and the like.

In some examples, oxygen saturation sensing device 58 may be configured to be placed on the skin of patient 6 to determine regional oxygen saturation of a particular tissue region, e.g., the frontal cortex or another cerebral location of patient 6. Oxygen saturation sensing device 58 may include emitter 62 and detector 64. Emitter 62 may include at least two light emitting diodes (LEDs), each configured to emit at different wavelengths of light, e.g., red or near infrared light. As used herein, the term “light” may refer to energy produced by radiative sources and may include any wavelength within one or more of the ultrasound, radio, microwave, millimeter wave, infrared, visible, ultraviolet, gamma ray or X-ray electromagnetic radiation spectra. In some examples, light drive circuitry (e.g., within sensing device 58, sensing circuitry 54, control circuitry 42, and/or processing circuitry 50) may provide a light drive signal to drive emitter 62 and to cause emitter 62 to emit light. In some examples, the LEDs of emitter 62 emit light in the range of about 600 nanometers (nm) to about 1000 nm. In a particular example, one LED of emitter 62 is configured to emit light at about 730 nm and the other LED of emitter 62 is configured to emit light at about 810 nm. Other wavelengths of light may be used in other examples.

Detector 64 may include a first detection element positioned relatively “close” (e.g., proximal) to emitter 62 and a second detection element positioned relatively “far” (e.g., distal) from emitter 62. In some examples, the first detection elements and the second detection elements may be chosen to be specifically sensitive to the chosen targeted energy spectrum of emitter 62. Light intensity of multiple wavelengths may be received at both the “close” and the “far” detector 64. For example, if two wavelengths are used, the two wavelengths may be contrasted at each location and the resulting signals may be contrasted to arrive at an oxygen saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). In operation, light may enter detector 64 after passing through the tissue of patient 6, including skin, bone, other shallow tissue (e.g., non-cerebral tissue and shallow cerebral tissue), and/or deep tissue (e.g., deep cerebral tissue). Detector 64 may convert the intensity of the received light into an electrical signal. The light intensity may be directly related to the absorbance and/or reflectance of light in the tissue. Surface data from the skin and skull may be subtracted out, to generate an oxygen saturation signal for the target tissues over time.

Oxygen saturation sensing device 58 may provide the oxygen saturation signal to processing circuitry 50. Additional example details of determining oxygen saturation based on light signals may be found in commonly assigned U.S. Pat. No. 9,861,317, which issued on Jan. 9, 2018, and is entitled “Methods and Systems for Determining Regional Blood Oxygen Saturation.” One example of such an oxygen saturation signal may be a plethysmography (PPG) signal.

In the example shown in FIG. 3, patient monitoring system 2 includes a blood pressure sensing device 60, which is configured to generate a blood pressure signal indicative of a blood pressure of patient 6. For example, blood pressure sensing device 60 may include a blood pressure cuff configured to non-invasively sense blood pressure or an arterial line configured to invasively monitoring blood pressure in an artery of patient 6. In some examples, the blood pressure signal may include at least a portion of a waveform of the acquisition blood pressure. Blood pressure sensing device 60 may be configured to generate a blood pressure signal indicative of the blood pressure of patient over time. Blood pressure sensing device 60 may provide the blood pressure signal to sensing circuitry 56, processing circuitry 50, or to any other suitable processing device, which may be part of patient monitoring system 2 or a device separate from patient monitoring system 2, such as another device co-located with patient monitoring system 2 or remotely located relative to patient monitoring system 2.

In operation, blood pressure sensing device 60 and oxygen saturation sensing device 58 may each be placed on the same or different parts of the body of patient 6. For example, blood pressure sensing device 60 and oxygen saturation sensing device 58 may be physically separate from each other and may be separately placed on patient 6. As another example, blood pressure sensing device 60 and oxygen saturation sensing device 58 may in some cases be supported by a single sensor housing. One or both of blood pressure sensing device 60 or oxygen saturation sensing device 58 may be further configured to measure other patient parameters, such as hemoglobin, respiratory rate, respiratory effort, heart rate, saturation pattern detection, response to stimulus such as bispectral index (BIS) or electromyography (EMG) response to electrical stimulus, or the like. While an example patient monitoring system 2 is shown in FIG. 3, the components illustrated in FIG. 3 are not intended to be limiting. Additional or alternative components and/or implementations may be used in other examples.

Processing circuitry 50 may be configured to receive one or more physiological signals generated by sensing devices 58 and 60 and sensing circuitry 54 and 56. The physiological signals may include a signal indicating blood pressure and/or a signal, such as a PPG signal, indicating oxygen saturation. Processing circuitry 50 may be configured to obtain the nociception parameter for patient 6 over time while patient 6 is in a medical procedure by continuously or periodically determining, based on the one or more physiological signals generated by sensing devices 58 and 60, the nociception parameter for patient 6. For example, the nociception parameter may be a value between 0 to 100 that indicates the amount of surgical stress experienced by patient 6 during the medical procedure. As processing circuitry 50 receives the one or more physiological signals during surgery of patient 6, processing circuitry 50 may be able to periodically or continuously determine, based on the one or more physiological signals, the nociception parameter for patient 6 over time. As such processing circuitry 50, sensing circuitry 54 and 56, and sensing devices 58 and may together implement nociception monitor 4 of patient monitoring system 2 shown in FIG. 1. In other examples, processing circuitry 50 may be configured to obtain the nociception parameter for patient 6 via one or more external devices. For example, processing circuitry 50 may be configured to communicate, via communication units 66, with an external device that sends the nociception parameter for patient 6 to processing circuitry 50.

In accordance with aspects of the present disclosure, processing circuitry 50 is configured to monitor the nociception parameter of patient 6 over time and to determine a characteristic nociception parameter at a point in time based on a plurality of values of the nociception parameter of patient 6 during a period of time, compare the characteristic nociception parameter with a nociception threshold to determine whether a nociception event has occurred at the point in time, and, in response to determining that a nociception event has occurred at the point in time, provide an indication to adjust the amount of analgesic administered to patient 6.

In some examples, processing circuitry 50 may be configured to determine the characteristic nociception parameter at a point in time as an average or as a weighted average of the values of the nociception parameter over the period of time. For example, processing circuitry 50 may be configured to weigh the values of the nociception parameter based on recency, so that more recent values are weighed more heavily in the weighted average, or based on noise metrics associated with the values of the nociception parameter, so that values associated with less noise are weighed more heavily in the weighted average.

In some examples, processing circuitry 50 may be configured to determine the characteristic nociception parameter at a point in time based at least in part on a most recently determined characteristic nociception parameter for patient 6. For example, processing circuitry 50 may be configured to determine the characteristic nociception parameter at a point in time as a weighted sum of the most recently determined characteristic nociception parameter for patient 6 and the value of the nociception parameter at the point in time. In this example, processing circuitry 50 may be configured to multiply the most recently determined characteristic nociception parameter for patient 6 with a weight w, multiply the value of the nociception parameter at the point in time with (1−w), and sum the result as the characteristic nociception parameter.

In some examples processing circuitry 50 may be configured to determine the characteristic nociception parameter at a point in time based at least in part on integrating (i.e., determining an integral of) the values of the nociception parameter that is greater than or equal to the nociception threshold over a period of time. For example, processing circuitry 50 may be configured to, for each value of the nociception parameter that is greater than or equal to the nociception threshold during the period of time, determine the difference between the value of the nociception parameter and the nociception threshold, and may be configured to sum the result to determine the characteristic nociception parameter at the point in time.

Processing circuitry 50 may be configured to compare the characteristic nociception parameter at the point in time with the nociception threshold to determine whether a nociception event has occurred at the point in time. If processing circuitry 50 determines that the characteristic nociception parameter is greater than or equal to the nociception threshold, then processing circuitry 50 may be configured to determine that a nociception event has occurred at the given point in time.

In some examples, processing circuitry 50 may be configured to suppress the determination that a nociception event has occurred at the point in time based at least in part on additional information. For example, if processing circuitry 50 detects noise in or associated with the nociception parameter that is above a noise threshold, processing circuitry may be configured to suppress the determination that a nociception event has occurred at the point in time even if the characteristic nociception parameter is greater than or equal to the nociception threshold.

In some examples, processing circuitry 50 may, in response to determining that the nociception event has occurred, output a notification via user interface 46. The notification can be any suitable visual, audible, somatosensory, or any combination thereof, notification that indicates the nociception event was detected. In some examples, the notification includes an indication to adjust an amount of analgesic to administer to patient 6. That is, processing circuitry 50 may cause analgesic administration device 18 to increase the amount of analgesic administered to patient 6 to dampen the surgical stress experienced by patient 6 by directly controlling analgesic administration device 18 or by generating a notification that causes a clinician to control analgesic administration device 18. Example analgesics that analgesic administration device 18 can administer include, but are not limited to, one or more of remifentanil, alfentanil, and fentanyl.

In some examples, to provide an indication to adjust an amount of analgesic to administer to patient 6, processing circuitry 50 may output, for display at display 16, an indication to increase an amount of analgesic to administer to patient 6, so that a clinician that views display 16 may therefore control analgesic administration device 18 to adjust the amount of analgesic administered to patient 6.

In some examples, to provide an indication to adjust an amount of analgesic to administer to patient 6, processing circuitry 50 may send, to analgesic administration device 18, the indication to adjust the amount of analgesic administered to patient 6. Analgesic administration device 18 may, in response to receiving the indication, adjust the amount of analgesic that analgesic administration device 18 delivers to patient 6. In this way, patient monitoring system 2 may act as an automated analgesic administration system.

In some examples, processing circuitry 50 may determine how much to adjust the amount of analgesic administered to patient 6 based on at least one of: a current amount of analgesic being administered to patient 6 and a total amount of analgesic administered to patient 6 during surgery. In some examples, it may be desirable to control the amount of analgesic being administered to patient 6 so that the amount at any point in time does not exceed a specified analgesic level. Thus, processing circuitry 50 may determine whether increasing the current amount of analgesic administered to patient 6 may cause the amount of analgesic administered to exceed the specified analgesic level and, if so, to reduce the increase in the amount of analgesic administered to patient 6 so that the amount of analgesic administered to patient 6 remains below the specified analgesic level.

In some examples, processing circuitry 50 may determine how much to adjust the amount of analgesic administered to patient 6 based on the integral of the values of the nociception parameter that is greater than or equal to the nociception threshold over a period of time because a higher integral value may indicate a higher level of nociception and indicate that more analgesia may be required. For example, if processing circuitry 50 is configured to determine the characteristic nociception parameter at the point in time based on the integral of the values of the nociception parameter that is greater than or equal to the nociception threshold over a period of time, then processing circuitry 50 may determine how much to increase the amount of analgesic administered to patient 6 based on the integral of the values of the nociception parameter that is greater than or equal to the nociception threshold over the period of time. For example, a higher value of the integral may indicate a relatively larger increase in the amount of analgesic to be administered to patient 6, while a lower value of the integral may indicate a relatively lesser increase in the amount of analgesic to be administered to patient 6. In some examples, memory 40 stores a table or other data structure that associates different values of the integral with different analgesia adjustment actions (e.g., different increases in dosages).

In some examples, it may be desirable to limit to the total amount of analgesic administered to patient 6 during surgery. Thus, in some examples, processing circuitry 50 may determine whether increasing the current amount of analgesic administered to patient 6 may cause the total amount of analgesic administered to patient 6 during surgery to exceed the limit and, if so, to reduce the increase in the amount of analgesic administered to patient 6 so that the amount of analgesic administered to patient 6 does not cause the total amount of analgesic administered to patient 6 during surgery to exceed the limit.

The components of patient monitoring system 2 that are shown and described as separate components are shown and described as such for illustrative purposes only. In some examples the functionality of some of the components may be combined in a single component. For example, the functionality of processing circuitry 50 and control circuitry 42 may be combined in a single processor system. Additionally, in some examples the functionality of some of the components of patient monitoring system 2 shown and described herein may be divided over multiple components or over multiple devices. For example, some or all of the functionality of control circuitry 42 may be performed in processing circuitry 50, or sensing circuitry 54 and 56. In other examples, the functionality of one or more of the components may be performed in a different order or may not be required.

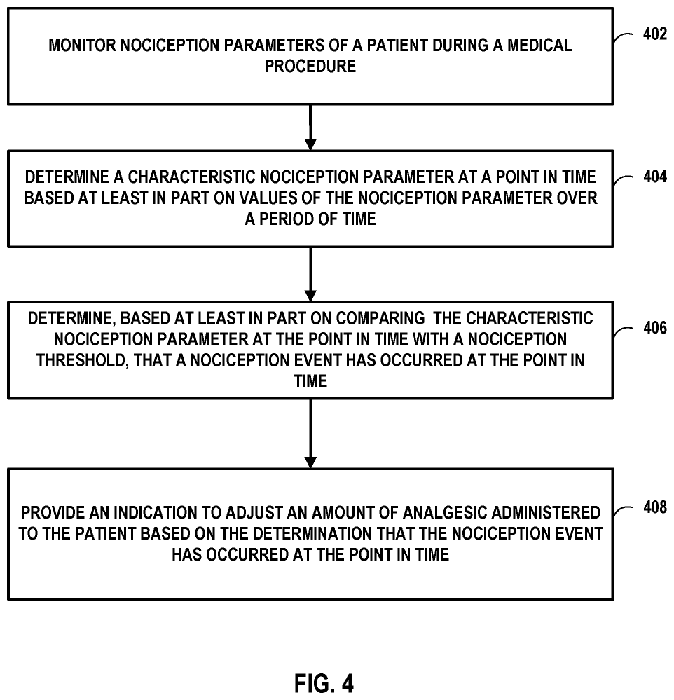

FIG. 4 is a flow diagram illustrating an example method of determining a patient-specific nociception threshold. Although FIG. 4 is described with respect to processing circuitry 50 of patient monitoring system 2 (FIGS. 1 and 3), in other examples, different processing circuitry, alone or in combination with processing circuitry 50, may perform any part of the technique of FIG. 4.

As shown in FIG. 4, processing circuitry 50 monitors nociception parameters of a patient 6 during a medical procedure (402). Processing circuitry 50 may determine a characteristic nociception parameter at a point in time based at least in part on values of the nociception parameter over a period of time (404). Processing circuitry 50 may determine, based at least in part on comparing the characteristic nociception parameter at the point in time with a nociception threshold, whether a nociception event has occurred at the point in time (406). Processing circuitry 50 may provide an indication to adjust an amount of analgesic administered to the patient 6 based on the determination that the nociception event has occurred at the point in time, wherein the determination is made based on a comparison between the characteristic nociception parameter at the point in time with a nociception threshold (408). For example, processing circuitry 50 may provide the indication in response to the determination that the nociception event has occurred at the point in time.

In some examples, to determine, based at least in part on comparing the characteristic nociception parameter at the point in time with the nociception threshold, the nociception event has occurred at the point in time, processing circuitry 50 further determine the characteristic nociception parameter at the point in time is greater than or equal to the nociception threshold, and, in response to determining that the characteristic nociception parameter is greater than or equal to the nociception threshold at the point in time, determines that the nociception event has occurred at the point in time.

In some examples, to determine the characteristic nociception parameter at the point in time based at least in part on values of the nociception parameter over the period of time, processing circuitry 50 further determines the characteristic nociception parameter at the point in time based on an average of the values of the nociception parameter over the period of time.

In some examples, to determine the characteristic nociception parameter at the point in time based at least in part on values of the nociception parameter over the period of time, processing circuitry 50 further determines the characteristic nociception parameter at the point in time based on a weighted average of the values of the nociception parameter over the period of time.

In some examples, to determine the characteristic nociception parameter at the point in time based on the weighted average of the values of the nociception parameter over the period of time, processing circuitry 50 further weighs the values of the nociception parameter over the period of time based at least in part on a recency of the values of the nociception parameter.

In some examples, to determine the characteristic nociception parameter at the point in time based on the weighted average of the values of the nociception parameter over the period of time, processing circuitry 50 further weighs the values of the nociception parameter over the period of time based at least in part on noise metrics associated with the values of the nociception parameter over the period of time.

In some examples, to determine the characteristic nociception parameter at the point in time based at least in part on values of the nociception parameter over the period of time, processing circuitry 50 further determines the characteristic nociception parameter at the point in time based at least in part on a most recently determined characteristic nociception parameter.

In some examples, to determine the characteristic nociception parameter at the point in time based at least in part on the most recently determined characteristic nociception parameter, processing circuitry 50 further determines the characteristic nociception parameter at the point in time based at least in part on the most recently determined characteristic nociception parameter and a value of the nociception parameter at the point in time.

In some examples, to determine the characteristic nociception parameter at the point in time based at least in part on the most recently determined characteristic nociception parameter and the value of the nociception parameter at the point in time, processing circuitry 50 further determines the characteristic nociception parameter at the point in time as a weighted sum of the most recently determined characteristic nociception parameter and the value of the nociception parameter at the point in time.

In some examples, to determine the characteristic nociception parameter at the point in time based at least in part on values of the nociception parameter over the period of time, processing circuitry 50 further determines the characteristic nociception parameter at the point in time based at least in part on an integral of the values of the nociception parameter over the period of time that are greater than or equal to the nociception threshold.

In some examples, to provide the indication to adjust an amount of analgesic administered to the patient, processing circuitry 50 further determines the amount of analgesic to administer to the patient based at least in part on an integral of the values of the nociception parameter over the period of time that are greater than or equal to the nociception threshold, and provide, an indication of the amount of analgesic to administer to the patient.

The following examples may illustrate one or more aspects of the disclosure.

Example 1: A method includes monitoring, by processing circuitry, a nociception parameter of a patient during a medical procedure; determining, by the processing circuitry, a characteristic nociception parameter at a point in time based at least in part on values of the nociception parameter over a period of time; determining, by the processing circuitry and based at least in part on comparing the characteristic nociception parameter at the point in time with a nociception threshold, a nociception event has occurred at the point in time; and providing, by the processing circuitry, an indication to adjust an amount of analgesic administered to the patient based on the determination that the nociception event has occurred at the point in time.

Example 2: The method of example 1, wherein determining, based at least in part on comparing the characteristic nociception parameter at the point in time with the nociception threshold, the nociception event has occurred at the point in time comprises: determining, by the processing circuitry, the characteristic nociception parameter at the point in time is greater than or equal to the nociception threshold; and in response to determining that the characteristic nociception parameter is greater than or equal to the nociception threshold at the point in time, determining, by the processing circuitry, that the nociception event has occurred at the point in time.

Example 3: The method of any of examples 1 or 2, wherein determining the characteristic nociception parameter at the point in time based at least in part on values of the nociception parameter over the period of time comprises: determining, by the processing circuitry, the characteristic nociception parameter at the point in time based on an average of the values of the nociception parameter over the period of time.

Example 4: The method of any of examples 1-3, wherein determining the characteristic nociception parameter at the point in time based at least in part on values of the nociception parameter over the period of time comprises: determining, by the processing circuitry, the characteristic nociception parameter at the point in time based on a weighted average of the values of the nociception parameter over the period of time.

Example 5: The method of example 4, wherein determining the characteristic nociception parameter at the point in time based on the weighted average of the values of the nociception parameter over the period of time comprises: weighing, by the processing circuitry, the values of the nociception parameter over the period of time based at least in part on a recency of the values of the nociception parameter.

Example 6: The method of example 4 or example 5, wherein determining the characteristic nociception parameter at the point in time based on the weighted average of the values of the nociception parameter over the period of time comprises: weighing, by the processing circuitry, the values of the nociception parameter over the period of time based at least in part on noise metrics associated with the values of the nociception parameter over the period of time.

Example 7: The method of any of examples 1-3, wherein determining the characteristic nociception parameter at the point in time based at least in part on values of the nociception parameter over the period of time comprises: determining, by the processing circuitry, the characteristic nociception parameter at the point in time based at least in part on a most recently determined characteristic nociception parameter.

Example 8: The method of example 7, wherein determining the characteristic nociception parameter at the point in time based at least in part on the most recently determined characteristic nociception parameter comprises: determining, by the processing circuitry, the characteristic nociception parameter at the point in time based at least in part on the most recently determined characteristic nociception parameter and a value of the nociception parameter at the point in time.

Example 9: The method of example 8, wherein determining the characteristic nociception parameter at the point in time based at least in part on the most recently determined characteristic nociception parameter and the value of the nociception parameter at the point in time comprises: determining, by the processing circuitry, the characteristic nociception parameter at the point in time as a weighted sum of the most recently determined characteristic nociception parameter and the value of the nociception parameter at the point in time.

Example 10: The method of any of examples 1-3, wherein determining the characteristic nociception parameter at the point in time based at least in part on values of the nociception parameter over the period of time comprises: determining, by the processing circuitry, the characteristic nociception parameter at the point in time based at least in part on an integral of the values of the nociception parameter over the period of time that are greater than or equal to the nociception threshold.

Example 11: The method of any of examples 1-10, wherein providing the indication to adjust an amount of analgesic administered to the patient comprises: determining, by the processing circuitry, the amount of analgesic to administer to the patient based at least in part on an integral of the values of the nociception parameter over the period of time that are greater than or equal to the nociception threshold; and providing, by the processing circuitry, an indication of the amount of analgesic to administer to the patient.

Example 12: A system includes memory; and processing circuitry configured to perform any combination of the method of claims 1-11.

Example 13: The system of example 12, further comprising sensing circuitry configured to sense the nociception parameter of the patient.

Example 14: The system of any of examples 12 or 13, further comprising an output device configured to output the indication to adjust the amount of analgesic administered to the patient.

Example 15: A non-transitory computer readable storage medium comprising instructions that, when executed, cause processing circuitry to perform any combination of the method of examples 1-11.

The techniques described in this disclosure, including those attributed to patient monitoring system 2, processing circuitry 50, control circuitry 42, sensing circuitries 54, 56, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

1. A system comprising:
memory configured to store a nociception threshold; and
processing circuitry configured to:
monitor a nociception parameter of a patient during a medical procedure, wherein the nociception parameter is based at least in part on one or more sensed physiological signals of the patient;
determine a characteristic nociception parameter at a point in time based at least in part on values of the nociception parameter over a period of time;
determine, based at least in part on comparing the characteristic nociception parameter at the point in time with the nociception threshold, a nociception event has occurred at the point in time; and
provide an indication to adjust an amount of analgesic administered to the patient based on the determination that the nociception event has occurred at the point in time.

2. The system of claim 1, wherein to determine, based at least in part on comparing the characteristic nociception parameter at the point in time with the nociception threshold, the nociception event has occurred at the point in time, the processing circuitry is further configured to:
determine the characteristic nociception parameter at the point in time is greater than or equal to the nociception threshold; and
in response to determining that the characteristic nociception parameter is greater than or equal to the nociception threshold at the point in time, determine that the nociception event has occurred at the point in time.

3. The system of claim 1, wherein to determine the characteristic nociception parameter at the point in time based at least in part on values of the nociception parameter over the period of time, the processing circuitry is further configured to:
determine the characteristic nociception parameter at the point in time based on an average of the values of the nociception parameter over the period of time.

4. The system of claim 1, wherein to determine the characteristic nociception parameter at the point in time based at least in part on values of the nociception parameter over the period of time, the processing circuitry is further configured to:
determine the characteristic nociception parameter at the point in time based on a weighted average of the values of the nociception parameter over the period of time.

5. The system of claim 4, wherein to determine the characteristic nociception parameter at the point in time based on the weighted average of the values of the nociception parameter over the period of time, the processing circuitry is further configured to:
weigh the values of the nociception parameter over the period of time based at least in part on a recency of the values of the nociception parameter.

6. The system of claim 4, wherein to determine the characteristic nociception parameter at the point in time based on the weighted average of the values of the nociception parameter over the period of time, the processing circuitry is further configured to:
weigh the values of the nociception parameter over the period of time based at least in part on noise metrics associated with the values of the nociception parameter over the period of time.

7. The system of claim 1, wherein to determine the characteristic nociception parameter at the point in time based at least in part on values of the nociception parameter over the period of time, the processing circuitry is further configured to:
determine the characteristic nociception parameter at the point in time based at least in part on a most recently determined characteristic nociception parameter.

8. The system of claim 7, wherein to determine the characteristic nociception parameter at the point in time based at least in part on the most recently determined characteristic nociception parameter, the processing circuitry is further configured to:
determine the characteristic nociception parameter at the point in time based at least in part on the most recently determined characteristic nociception parameter and a value of the nociception parameter at the point in time.

9. The system of claim 8, wherein to determine the characteristic nociception parameter at the point in time based at least in part on the most recently determined characteristic nociception parameter and the value of the nociception parameter at the point in time, the processing circuitry is further configured to:
determine the characteristic nociception parameter at the point in time as a weighted sum of the most recently determined characteristic nociception parameter and the value of the nociception parameter at the point in time.

10. The system of claim 1, wherein to determine the characteristic nociception parameter at the point in time based at least in part on values of the nociception parameter over the period of time, the processing circuitry is further configured to:
determine the characteristic nociception parameter at the point in time based at least in part on an integral of the values of the nociception parameter over the period of time that are greater than or equal to the nociception threshold.

11. The system of claim 1, wherein to provide the indication to adjust an amount of analgesic administered to the patient, the processing circuitry is further configured to:
determine the amount of analgesic to administer to the patient based at least in part on an integral of the values of the nociception parameter over the period of time that are greater than or equal to the nociception threshold; and
provide, an indication of the amount of analgesic to administer to the patient.

12. A method comprising:
monitoring, by processing circuitry, a nociception parameter of a patient during a medical procedure, wherein the nociception parameter is based at least in part on one or more sensed physiological signals of the patient;
determining, by the processing circuitry, a characteristic nociception parameter at a point in time based at least in part on values of the nociception parameter over a period of time;
determining, by the processing circuitry and based at least in part on comparing the characteristic nociception parameter at the point in time with a nociception threshold, a nociception event has occurred at the point in time; and
providing, by the processing circuitry, an indication to adjust an amount of analgesic administered to the patient based on the determination that the nociception event has occurred at the point in time, wherein the determination is made based on a comparison between the characteristic nociception parameter at the point in time with a nociception threshold.

13. The method of claim 12, wherein determining, based at least in part on comparing the characteristic nociception parameter at the point in time with the nociception threshold, the nociception event has occurred at the point in time comprises:
determining, by the processing circuitry, the characteristic nociception parameter at the point in time is greater than or equal to the nociception threshold; and
in response to determining that the characteristic nociception parameter is greater than or equal to the nociception threshold at the point in time, determining, by the processing circuitry, that the nociception event has occurred at the point in time.

14. The method of claim 12, wherein determining the characteristic nociception parameter at the point in time based at least in part on values of the nociception parameter over the period of time comprises:
determining, by the processing circuitry, the characteristic nociception parameter at the point in time based on an average of the values of the nociception parameter over the period of time.

15. The method of claim 14, wherein the average is a weighted average, and wherein determining the characteristic nociception parameter at the point in time based on the weighted average of the values of the nociception parameter over the period of time comprises:
weighing, by the processing circuitry, the values of the nociception parameter over the period of time based at least in part on a recency of the values of the nociception parameter.

16. The method of claim 14, wherein the average is a weighted average, and wherein determining the characteristic nociception parameter at the point in time based on the weighted average of the values of the nociception parameter over the period of time comprises:
weighing, by the processing circuitry, the values of the nociception parameter over the period of time based at least in part on noise metrics associated with the values of the nociception parameter over the period of time.

17. The method of claim 12, wherein determining the characteristic nociception parameter at the point in time based at least in part on values of the nociception parameter over the period of time comprises:
determining, by the processing circuitry, the characteristic nociception parameter at the point in time based at least in part on a most recently determined characteristic nociception parameter.

18. The method of claim 17, wherein determining the characteristic nociception parameter at the point in time based at least in part on the most recently determined characteristic nociception parameter comprises:
determining, by the processing circuitry, the characteristic nociception parameter at the point in time as a weighted sum of the most recently determined characteristic nociception parameter and a value of the nociception parameter at the point in time.

19. The method of claim 12, wherein determining the characteristic nociception parameter at the point in time based at least in part on values of the nociception parameter over the period of time comprises:
determining, by the processing circuitry, the characteristic nociception parameter at the point in time based at least in part on an integral of the values of the nociception parameter over the period of time that are greater than or equal to the nociception threshold.

20. A non-transitory computer readable storage medium comprising instructions that, when executed, cause processing circuitry to:
monitor a nociception parameter of a patient during a medical procedure, wherein the nociception parameter is based at least in part on one or more sensed physiological signals of the patient;
determine a characteristic nociception parameter at a point in time based at least in part on values of the nociception parameter over a period of time;
determine, based at least in part on comparing the characteristic nociception parameter at the point in time with a nociception threshold, a nociception event has occurred at the point in time; and
provide an indication to adjust an amount of analgesic administered to the patient based on the determination that the nociception event has occurred at the point in time, wherein the determination is made based on a comparison between the characteristic nociception parameter at the point in time with a nociception threshold.
